SEQUENCE LISTING (1 sequence)

SEQ ID NO: 1 - protein, 509 aa, Homo sapiens
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRP

(19) United States
(12) Patent Application Publication
(10) Pub. No.: US 2017/0197011 A1
(43) Pub. Date: Jul. 13, 2017

(54) METHODS FOR REPAIRING CARTILAGE DAMAGE

(71) Applicant: VIVOSCRIPT, INC., Costa Mesa, CA (US)

(72) Inventors: Cong-Qiu CHU, Portland, OR (US); Yong ZHU, Anaheim, CA (US); Shili WU, Edmond, OK (US); Jun BAO, Rowland Heights, CA (US)

(21) Appl. No.: 15/313,965

(22) PCT Filed: Jun. 5, 2015
(86) PCT No.: PCT/US15/34309

§ 371 Date: Nov. 24, 2016

Related U.S. Application Data
(60) Provisional application No. 62/008,513, filed on Jun. 6, 2014.

(51) Int. Cl. A61L 27/38; A61K 9/00; A61L 27/54; A61L 27/24; A61K 38/17; C07K 14/47

(52) U.S. Cl. CPC A61L 27/3852; A61K 38/1709; C07K 14/47; A61L 27/54; A61L 27/24; A61K 9/0024; C07K 2319/095; C07K 2319/60; C07K 2319/10; A61L 2430/06; A61L 2300/412

(57) ABSTRACT

A method for repairing cartilage damage comprising (a) creating a microfracture or performing other bone marrow stimulation techniques on a patient inflicted with cartilage damage; and (b) administering a composition to the microfracture, wherein the composition comprises an agent capable of regenerating organized hyaline cartilage.

### CROSS-REFERENCE TO RELATED APPLICATIONS

This application claims priority to U.S. provisional patent application No. 62/008,513, filed Jun. 6, 2014, the disclosure of which is incorporated herein by reference in its entirety.

### FIELD OF THE INVENTION

The present invention generally relates to methods for repairing cartilage damage.

### BACKGROUND OF THE INVENTION

Articular cartilage is a highly organized tissue with low cell density and limited nutrient supply. Once it is damaged by trauma or degenerative arthritis, it has a limited capacity for regeneration. The most common joint disorder, osteoarthritis (OA), is afflicting millions of people with symptoms including severe pain, swelling and clicking of joints. To make things worse, OA cannot be cured—only its symptoms can be controlled.

OA is the most common form of arthritis and the fourth leading cause of disability worldwide. Over 70% of Americans between the ages of 55 and 70 are estimated to be affected by OA. Treatment of OA has remained to be a daunting challenge and bears a substantial burden to the health care system. Current treatment consists essentially of palliative pain relief and physical therapy which do not change the disease course, and most patients with OA will progress to advanced stage and require total joint replacement. OA is characterized by progressive breakdown of articular cartilage, and ultimately leads to functional failure of synovial joints. Regeneration of cartilage has been an attractive approach to OA therapy. Since hyaline cartilage cannot spontaneously regenerate in vivo, strategy for repairing articular cartilage is to fill a gap with cartilage transplants or a tissue-engineered cartilage like tissue, or to stimulate progenitor cells to differentiate into chondrocytes in situ. Although successful repair is reported with autologous cartilage transplants, significant drawbacks are associated with this procedure. Autologous cartilage transplants require donor tissue from non- or less-weight bearing area of articular cartilage which is limited in supply and leads to new morbidity to the donor site. In vitro expansion of chondrocytes may cause de-differentiation of chondrocytes and insufficient cell supply. In vitro matrix-assisted tissue engineering involves long term cell and multiple surgical procedures. For these reasons, in situ regeneration of cartilage is a highly desirable strategy to repair the defective articular cartilage. A commonly practiced procedure for cartilage repair, microfracture induces migration of bone marrow mesenchymal stem cells (MSCs) to the site of cartilage defect and promotes fibrocartilage production (FIG. 1A). It involves drilling small holes into the subchondral bone marrow space which underlies regions of damaged cartilage, inducing bleeding at the defect site and allowing for the formation of a blood clot. The clot contains multipotent MSCs from the bone marrow, which have the potential to differentiate into chondroblasts and chondrocytes. Microfracture is an easy, simple, minimally invasive and single stage procedure with low morbidity and low cost.

However, like other surgical procedures, the cartilage formed is fibro-cartilaginous. Fibrocartilage is non-durable and functionally inadequate in the long-term. Fibrocartilage has poor resistance to shear forces, in contrast to hyaline cartilage. Under normal physiological conditions, hyaline cartilage provides shock absorption and lubrication in diarthrodial joints as articular cartilage. A highly organized tissue, hyaline cartilage is substantially durable, attributable to its extracellular matrix produced by chondrocytes and consisting of collagen fibrils composed of types II, IX, and XI collagen molecules, proteoglycans, and other matrix proteins.

Hyaline cartilage has a poor intrinsic capacity for healing. A type of scar tissue, fibrocartilage expresses types I and II collagen; hyaline cartilage, in contrast, does not express type I collagen. As the presence of type I collagen impairs cartilage-specific matrix architecture and mechanical function, repair of cartilage damage by fibrocartilage leads to morbidity and functional impairment. Thus, the goal for repair of cartilage injury is to regenerate organized hyaline cartilage. Healing of cartilage damage with hyaline cartilage rather than fibrocartilage remains a challenging clinical problem. Therefore, there remains a continuing need for methods for repairing cartilage damage by regenerating organized hyaline cartilage.

### SUMMARY OF THE INVENTION

The present application provides a method for repairing cartilage damage, and a composition used thereof.

In certain embodiments, the method comprises (a) creating a microfracture or performing other bone marrow stimulation techniques on a patient inflicted with cartilage damage; and (b) administering a composition to the microfracture site, wherein the composition comprises an agent capable of regenerating organized hyaline cartilage.

In some embodiments, the agent is capable of inducing mesenchymal stem cells (MSCs) to differentiate into chondrocytes. In some embodiments, the agent is a polypeptide. In some embodiments, the agent is a polypeptide comprising an effector domain. In some embodiments, the effector domain is a chondrogenic transcription factor. Preferably, the chondrogenic transcription factor is SOX9. In certain embodiments, the transcription factor is a variant of SOX9 having an enhanced cell-penetrating peptide. In certain embodiments, the transcription factor is a variant of SOX9 having a disrupted nuclear export peptide.

In some embodiments, the agent is a nucleic acid. In some embodiments, the agent is a nucleic acid encoding a polypeptide comprising a chondrogenic transcription factor. Preferably, the chondrogenic transcription factor is SOX9.

In some embodiments, the agent is a compound or a small molecule.

In some embodiments, the agent is capable of stimulating the expression of SOX9. Such agent includes insulin like growth factor 1 (IGF-1), fibroblast growth factor-2 (FGF-2), bone morphogenetic proteins (BMPs), transforming growth factor-β (TGF-β).

In some embodiments, the agent needs to be in the nuclei to be functional. Therefore, in some embodiments, the agent further comprises a transduction domain to facilitate the agent to penetrate the cell membrane and get into cell nuclei. The transduction domain is capable of translocating the transcription factors into cells or even nuclei. In some embodiment, the transduction domain is selected from the group consisting of TAT, Poly-arginine, Penetratin (Antennapedia), VP22, Transportan, MAP, MTS and PEP-1. In the case that the agent is a polypeptide, the transduction domain can be fused to the N-termini or the C-termini of the polypeptide. In some embodiments, the agent comprises nuclear localization signals which can help it get into nuclei.

In some embodiment, the agent comprises a supercharged peptide to be transducible. In some embodiment, the supercharged peptide is supercharged GFP.

In some embodiment, the agent is made transducible, after being modified or mutated from their natural sequences to supercharged forms, or other transducible formats.

In some embodiment, the agent is modified to comprise a peptide that is a ligand for some cell-surface receptors and will facilitate the entry of the agent into cells through receptor mediated endocytosis.

In some embodiment, the composition being administered to the microfracture site further comprises a carrier. In some embodiment, the carrier is a polymer or a protein transducible domain PTD peptide. In some embodiment, the carrier is a collagen membrane or other biocompatible, resorbable membranes, or biocompatible matrices.

In some embodiments, the agent is from natural sources. In some embodiments, the agent is produced from E. coli or other expression systems using recombinant DNA technology, or synthesized.

In some embodiment, the method further comprising (c) administering the patient an immune suppressor.

In some embodiment, the composition is administered to the microfracture site by loading the agent to a carrier, such as a collagen membrane. Then the carrier is placed and/or secured on the surface of microfracture sites during the procedure.

In some embodiment, the composition is administered by directly injecting the composition into the synovial cavity of the microfracture on a patient.

In another aspect, the present application provides a composition for repairing cartilage damage. In certain embodiments, the composition comprises an agent capable of regenerating organized hyaline cartilage as described supra. In certain embodiments, the composition further comprises a carrier. In some embodiments, the carrier is a collagen membrane.

### BRIEF DESCRIPTION OF THE FIGURES

FIG. 1A. Schematic diagram of cartilage repair using microfracture only.

FIG. 1B. Schematic diagram of cartilage repair using microfracture in combination with a composition comprising an agent (e.g., transducible or cell-penetrating SOX9) capable of inducing hyaline cartilage.

FIG. 2. Mesenchymal stem cells (MSCs) are multipotent and may differentiate to one of several types of cells. Expression or transduction of Sox9 protein will stimulate MSC proliferation and direct MSCs towards the chondrocyte fate.

FIG. 3. Location of nuclear-localization signals of SOX9 HMG. The high-mobility group (HMG) domain of SOX9 contains two nuclear-localization signals (NLS). The N-terminal NLS (nNLS) binds calmodulin (CaM) while the C-terminal NLS (cNLS) binds importin-b (Impb). Cam and Impb are two proteins involved in transporting a wide range of protein through nuclear pore complexes. Thus, SOX9 is actively transported into nucleus via both.

FIG. 4. scSOX9 (supercharged GFP fused with SOX9, or supercharged SOX9) induced increased collagen type II but decreased collagen type I and X expression. Human bone marrow derived MSC were cultured with DMEM containing 1% FBS and high glucose (4.5 g/1) with addition of buffer only or 10 μg/ml of scSOX9. At the indicated time point, RNA was extracted and RT-PCR was performed with TaqMan probe based analysis assay for collagen (Col) type I, II and X mRNA expression. Collagen mRNA expression was relative to GAPDH and scSOX9 treated was compared to buffered treated (n=3 in each time points).

FIG. 5. scSOX9 induced collagen type II expression. Human bone marrow derived MSC was cultured with 10 μg/ml of scGFP (supercharged GFP) (A and C) or scSOX9 (B and D) in monolayer (A and B) or aggregate (C and D) for chondrogenesis. At day 14, aggregates were harvested and snap-frozen. Cryostat sections were stained with a mouse anti-human collagen type II monoclonal antibody. (Immunoperoxidase staining. Note the poorly formed aggregate by MSC cultured with scGFP only. Representative of 3 experiments.

FIG. 6. Efficiency of scSOX9 delivery into MSC in vivo. Cartilage defect was created and microfracture was performed at the patellar groove of the femur in the knee joints of New Zealand female rabbits. One hour after administration of scSOX9, the bone marrow clot was harvested and digested. The cell suspension was washed with PBS containing 20 units of heparin to wash off possible cell membrane bound scSOX9, then stained with a cocktail of monoclonal antibodies including PE labeled CD11b, CD79a, MHC-DR and APC labeled CD90, and analyzed in flow cytometry. MSC was defined as CD90+/CD11b−/CD79a−/DR−. GFP positive cells indicated that scSOX9 entered MSCs. The figure represents dot plot and histogram of three experiments.

FIG. 7. Schematic diagram of administering an agent (e.g., supercharged SOX9) through a carrier (e.g., collegen membrane) to microfracture site for cartilage repair.

FIG. 8. Assessment of cartilage repair. Gross appearance (top panel): Photographs of rabbit knee articular cartilage defects 8 weeks after treatments, the joint were examined grossly (A-D). The dotted circles indicate the original defect margin. Histological analysis (middle panel): The distal femurs were fixed in 10% formalin, decalcified, embedded in paraffin, and cut into 5 μm sections. Sections from each sample were then stained with hematoxylin and eosin for morphological evaluation (E-H). Safranin O and fast green staining (bottom panel) for glycosaminoglycan distribution (I-L). The arrows indicate defect margin. N=3 in each group.

FIG. 9. High magnification histology. Upper panel for H-E staining; lower panel for Safranin O staining.

FIG. 10. Screening test to select the best SOX9 variants.

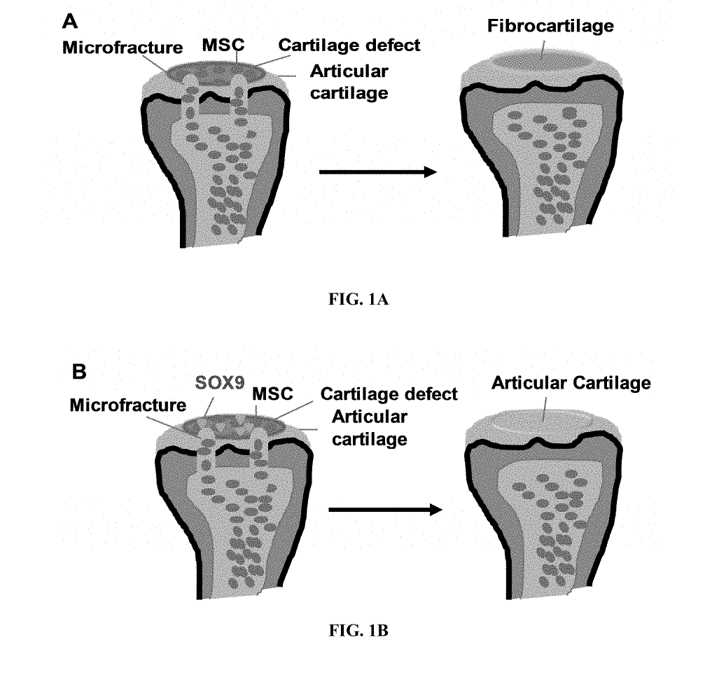

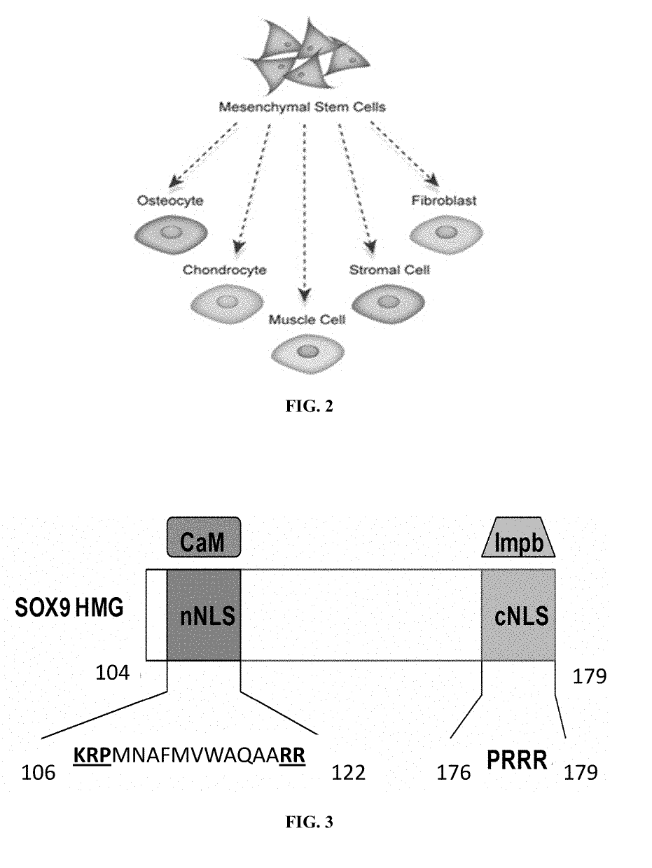

### DETAILED DESCRIPTION OF THE INVENTION

In one aspect, the present application provides a method for repairing cartilage damage. The method can be used to repair both fresh cartilage injury as well as aged injury and to treat OA derived from cartilage injury. This procedure will halt the progression of cartilage injury and progression to OA and ultimately will delay the requirement of joint replacement in patients with OA. In certain embodiments, the method comprises the steps of (a) creating a microfracture or performing other bone marrow stimulation techniques on a patient inflicted with cartilage damage; and (b) administering a composition to the site of the microfracture, wherein the composition comprises an agent capable of regenerating organized hyaline cartilage.

The procedure of microfracture surgery is known in the art. In principle, microfracture surgery creates in the underlying bone tiny fractures, from which blood and bone marrow seep out to create a blood clot that releases cartilage-building cells. Generally, the base of the defective cartilage location is shaved or scraped to induce bleeding. An arthroscopic awl or pick is then used to make small holes in the subchondral bone plate. The end of the awl is manually struck with a mallet to form the holes while care is made not to penetrate too deeply and damage the subchondral plate. The holes penetrate a vascularization zone and stimulate the formation of a fibrin clot containing pluripotent stem cells.

By drilling small holes deep into the subchondral bone marrow space, microfracture induces bleeding of bone marrow and forms clot at the surface of cartilage defect. Some of the MSCs contained in the bone marrow clot then differentiate into chondrocytes. Clinical studies indicate that microfracture provides effective short-term improvement of joint function but is with shortcomings of poor long-term improvement and possible functional deterioration after 24 months. This is mainly because of the low quality of fibrocartilage or fibrohyaline hybrid tissue generated by this procedure. Fibrocartilage contains less proteoglycan and more type I collagen with inferior mechanical property.

The main reason for forming mainly fibrocartilage via the microfracture procedure is the multipotency of MSCs: they can differentiate not only to chondrocytes, but also to osteocytes, muscle cycles, stromal cells, or fibroblasts. In the microfracture procedure, a significant percentage of MSCs turn into stromal cells and fibroblasts, resulting in the formation of fibrocartilage. The present application provides a method of modifying the microfracture procedure by adding some chondrogenic composition that directs MSCs towards chondrocytes pathway only, so that hyaline cartilage will be produced.

In certain embodiments, the composition comprises an agent capable of inducing mesenchymal stem cells to differentiate into chondroblasts and/or chondrocytes. In certain embodiments, the agent is selected from the group consisting of TGF-β-1, 2, and 3, BMP-2-4-7, CDMP, GDF-5, IGF-1, FGF family, SMAD-1, -2, -3, -4, -5, -6, -7, -8, EGF, PDGF, type II collagen, type IX collagen, cartilage-link protein, SOX5, SOX6, SOX9, MEF2C, Dlx5, Nkx2.5, PTHrP, Ihh, Wnt and CTGF.

In some embodiment, the agent is made transducible, after being modified or mutated from their natural sequences to supercharged forms, or other transducible formats.

In certain embodiments, the agent is a nucleic acid. In certain embodiments, the nucleic acid encodes a polypeptide comprising a chondrogenic transcription factor.

In certain embodiments, the agent is a compound or a small molecule. In certain embodiments, the agent stimulates the expression of SOX9.

In some preferred embodiments, the agent comprises the transcription factor SOX9 in a transducible or cell-penetrating format. SOX9 belongs to the Sox (Sry-type HMG box) family and has been identified as a “master regulator” of the chondrocyte phenotype. Effects of SOX9 on MSCs are two-fold: stimulating proliferation and promoting differentiation into chondrocytes. The amino acid sequence of human SOX9 protein (SEQ ID NO. 1) can be found in National Center for Biotechnology Information (NCBI) database with GenBank No.: CAA86598.1.

In more preferred embodiments, the agent comprises a variant of SOX9 that has an enhanced cell-penetrating peptide (CPP). In certain embodiments, the enhanced cell-penetrating peptide is endogenous. In certain embodiments, the CPP has the sequence ₁₇₄X¹QPRRRKX²X³K₁₈₃, wherein X¹ is Y, K or R, X² is S or R, X³ is V or K, the number represents the amino acid residues in human SOX9 protein sequence (SEQ ID NO. 1). In certain embodiments, X¹ is K or R, X² is R, X³ is K

In certain embodiments, the variant of SOX9 has a disrupted nuclear export sequence (NES). In certain embodiments, the NES is ₁₃₄ELSKTLGKLWRLL₁₄₆, wherein the number represents the amino acid residues in human SOX9 protein sequence (SEQ ID NO. 1). In certain embodiments, the disrupted NES has a mutation of L142A.

In some embodiments, the agent needs to be in the nuclei to be functional. Therefore, in some embodiments, the agent further comprises a transduction domain to facilitate the agent to penetrate the cell membrane and get into cell nuclei. The transduction domain is capable of translocating the transcription factors into cells or even nuclei. Examples of a transduction domain has been disclosed in PCT Application PCT/US2009/069518, published as WO2010075575, which is incorporated herein by reference in their entirety. Examples of a transduction domain include, without limitation, polymers such as cationic lipid polymers and nanoparticles, protein transduction domains (PTD), cell penetrating peptides (CPP1), cell permeating peptides (CPP2), activatable cell penetrating peptides or conjugates (ACPP), and cell-targeting peptides (CTP).

In some embodiment, the transduction domain is selected from the group consisting of TAT, Poly-arginine, Penetratin (Antennapedia), VP22, Transportan, MAP, MTS and PEP-1. In the case that the agent is a polypeptide, the transduction domain can be fused to the N-termini or the C-termini of the polypeptide. In some embodiments, the agent comprises nuclear localization signals which can help it get into nuclei.

In some embodiment, the agent comprises a supercharged peptide to be transducible. In some embodiment, the supercharged peptide is supercharged GFP.

In some embodiment, the agent is modified to comprise a peptide that is a ligand for some cell-surface receptors and will facilitate the entry of the agent into cells through receptor mediated endocytosis.

In some embodiment, the composition being administered to the microfracture site further comprises a carrier. In some embodiment, the carrier is a polymer or a protein transducible domain PTD peptide. In some embodiment, the carrier is a collagen membrane or other biocompatible, resorbable membranes. In some embodiment, the carrier is a matrix, including hydrogel and fibrin.

In some embodiments, the agent is from natural sources. In some embodiments, the agent is produced from E. coli or other expression systems using recombinant DNA technology, or synthesized.

In some embodiment, the method further comprising (c) administering the patient an immune suppressor. Examples of immune suppressor include, without limitation, glucocorticoids, cytostatics, antibodies (e.g., anti-CD20 antibodies, anti-CD25 antibodies), drugs acting on immunophilins (e.g., ciclosporin, tacrolimus, sirolimus), interferons, opioids, mycophenolate,

In some embodiment, the composition is administered to the microfracture site by loading the agent to a carrier, such as a collagen membrane. Then the carrier is placed on the surface of microfracture sites during the procedure.

In some embodiment, the composition is administered by directly injecting the composition into the synovial cavity of the microfracture on a patient.

In another aspect, the present application provides a composition for repairing cartilage damage. In certain embodiments, the composition comprises an agent capable of regenerating organized hyaline cartilage as disclosed supra. In certain embodiments, the composition further comprises a carrier. In some embodiments, the carrier is a collagen membrane.

### EXAMPLES

The following examples are presented to illustrate the present invention. They are not intended to be limiting in any manner.

### Example 1

Super-charged SOX9 (scSOX9) comprising SOX9 protein fused with super-charged green fluorescence protein (scGFP) can penetrate MSCs in vitro.

Method

Commercial human MSCs (ScienCell Research Laboratories) at passage 5 were maintained and expanded in culture medium in sub-confluence condition. Induction of MSC differentiation was carried out in high-throughput cell aggregate culture as described. 2.5×10⁵ cells/well MSC cells in 0.2 ml are cultured in V-bottomed polypropylene 96-well plates. For positive control, MSCs were cultured in DMEM-HG supplemented with 10% ITS+Premix Tissue Culture Supplement (Becton Dickson), 10⁻⁷ M dexamethasone and 10 ng/ml TGF-β1. Under this culture condition, MSCs undergo chondrogenic differentiation within 2-3 weeks, producing abundant extracellular matrix composed primarily of cartilage-specific molecules such as type II collagen and aggrecan. The expression of these cartilage markers were used as evidence of the chondrogenic differentiation of MSCs. To test the capacity of SOX9 variants in inducing chondrogenesis, MSCs were cultured with in DMEM-HG medium containing each protein at concentration of 1-20 μg/l without the supplement of cocktail of growth factors. Original scSOX9 was served as a positive control and native SOX9 protein was used as negative control. Cell aggregates were harvested at week 1, week 2 and week 3 to determine expression of matrix proteins and morphology.

The initial cell aggregates contained type I collagen but no cartilage-specific molecules. By week 1, collagen type II was detectable and throughout the cell aggregate. Type X collagen initially expressed and then was down regulated at later times by scSOX9. Collagen type I, II and X mRNA expression were determined using qPCR with TagMan primers and probes (collagen type I COL1A1 Hs00164004_m1, COL2A1 Hs00264051 ml, COL10A1 Hs00166657_m1 and aggrecan Hs00153936 ml ACAN, Life Technologies). Collagen expression at protein level was determined using immunohisotochemical staining on cryostat sections with anti-collagen type I antibody (clone col-1, Sigma), anti-collagen type II antibody (Developmental Studies Hybridoma Bank, University of Iowa) and anti-collagen type X antibody (gift from Dr. Gary Gibson, Henry Ford Hospital & Medical Center).

Glycosaminoglycans (GAG) are essential extracellular molecules of cartilage. GAG content was quantified using a modified Safranin-O dye-assay as described. Regenerated tissues are digested by papain. The digested samples were added to Safranin-O dye agent on nitrocellulose membrane in a dot-blot apparatus. Reaction was measured by absorbance at 536 nm against standard curve of chondroitin sulfate C prepared from shark cartilage.

Toluidine blue staining was performed to assess the content of aggrecan/proteoglycan in the cell aggregate.

Results

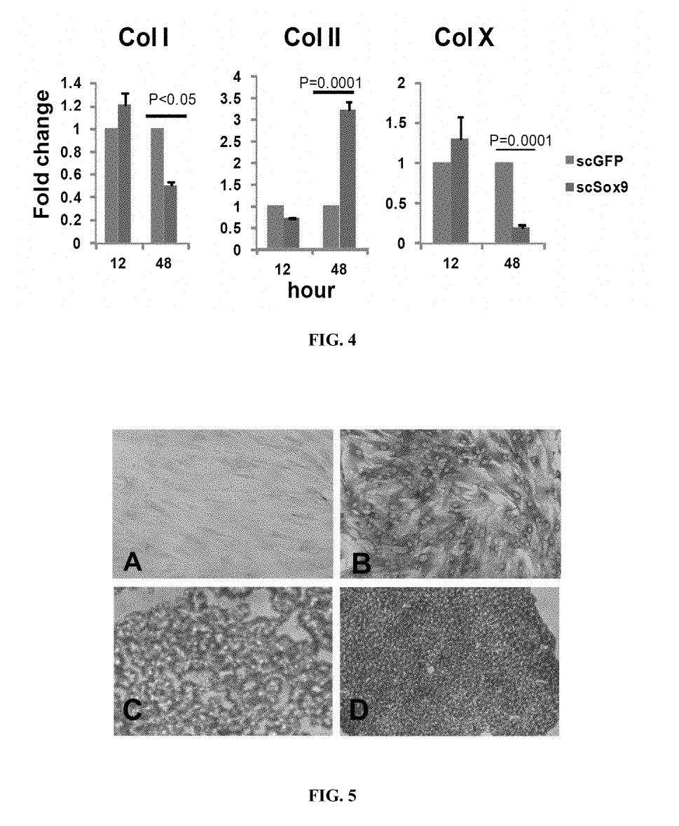

The bioactivity of scSOX9 was tested for induction of MSC chondrogenic differentiation using a well-established in vitro culture system in monolayer and cell aggregates. scSOX9 induced chondrogenesis of MSC was compared with that induced by mixture of growth factors in culture as described. Human bone marrow derived MSC at passage 5 were cultured in Dulbecco's Modified Eagle's Medium (DMEM) with high glucose (4.5 g/1) (DMEM-HG). In the standard protocol, DMEM-HG was supplemented with 10% ITS+Premix Tissue Culture Supplement (Becton Dickson), 10⁻⁷ M dexamethasone and 10 ng/ml transforming growth factor (TGF)-β1. In scSOX9 induced chondrogenesis protocol, scSOX9 was added in DMEM-HG to substitute for the supplement of all growth factors. scSOX9 alone without addition of other growth factors was capable of inducing MSC chondrogenesis, similar to that induced by the cocktail of growth factors in the standard protocol. As early as 48 hours, scSOX9-treated MSC started to change morphology into chondrocyte like cells and this morphology maintained for at least 21 days in culture. The positive staining with toluidine blue for aggrecan demonstrated that these morphologically changed cells functioned like chondrocytes. Furthermore, while inducing MSC morphology change, scSOX9 also induced increased collagen type II expression and downregulated collagen type I and type X production (FIG. 4 and FIG. 5). This composition of matrix proteins is typical characteristics of articular chondrocytes.

### Example 2

Super-charged SOX9 (scSOX9) comprising SOX9 protein fused with super-charged green fluorescence protein (scGFP) can induce chondrogenesis in vivo.

The release of scSOX9 from carrier was tested. A commercial bilayer collagen membrane (Bio-Gide) was used to serve as a carrier for scSOX9 to be administered at the site of microfracture. A Bio-Gide membrane at 4 mm in diameter was soaked in 100 μg/ml of scSOX9 solution for one hour. Green fluorescence was grossly visible on the Bio-Gide membrane, and 60% of the total scSOX9 was carried. Release of scSOX9 from Bio-Gide membrane was tested by rinsing the membrane with PBS containing 20 U/ml heparin (pH7.4) for 1 hour, over 95% of scSOX9 bound on Bio-Gide membrane was released and re-dissolved in solution.

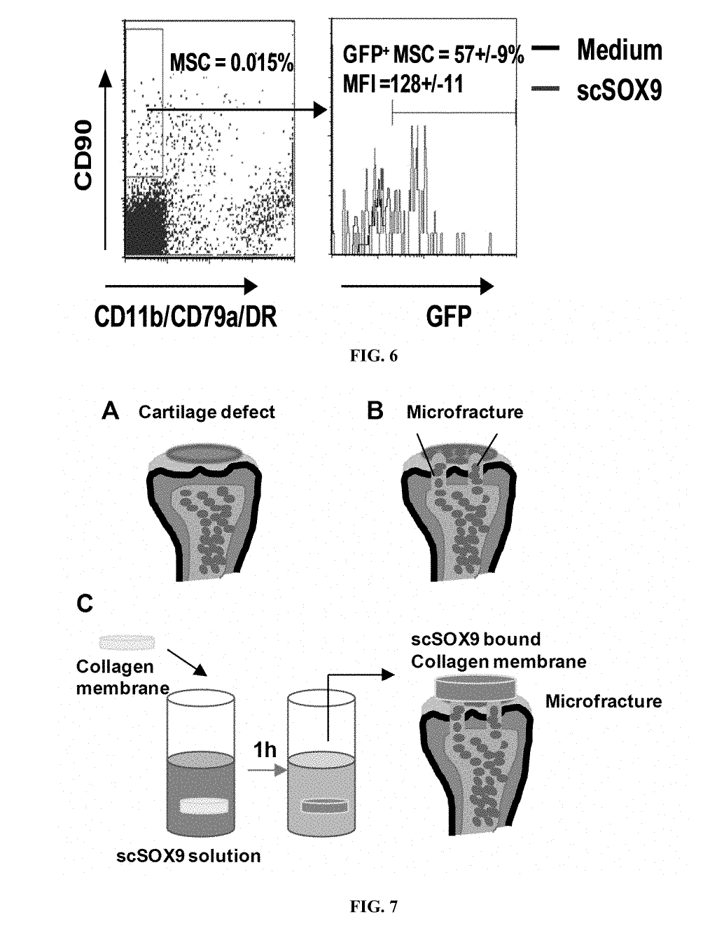

The efficiency of scSOX9 delivery into MSC in vivo was assessed. A cylindrical cartilage defect of 4 mm in diameter and 3 mm in depth was created in patellar groove of the femur of New Zealand female rabbits. Microfracture was created using 0.9 mm Kirschner wire and bleeding of bone marrow was allowed to fully fill the cartilage defect. A Bio-Gide membrane harboring scSOX9 was secured to cover the defect. One hour later, the bone marrow clot from the defect was harvested and minced and digested with streptokinase. An average of 30 ml of bone marrow clot was recovered from each defect (the calculated volume of each defect was 37.68 ml). The digested bone marrow cell suspension was washed with PBS containing 20 units/ml of heparin to eliminate cell membrane bound scSOX9, and stained with antibodies against CD90-APC, CD11b-PE, CD79a-PE and MHC-DR-PE (Ad Serotec) for 30 minutes. After red blood cells were lysed the cells were analyzed on flow cytometry for delivery of scSOX9 into MSC. As shown in the FIG. 6, MSC were defined as CD90+/CD11b−/CD79a−/DR− and were comprised of about 0.015% of the total nucleated bone marrow cells. The frequency of MSC in our bone marrow cell preparation was consistent with previously estimated. About 60% of MSC in the bone marrow clot were showing GFP positive, indicating that scSOX9 entered these cells

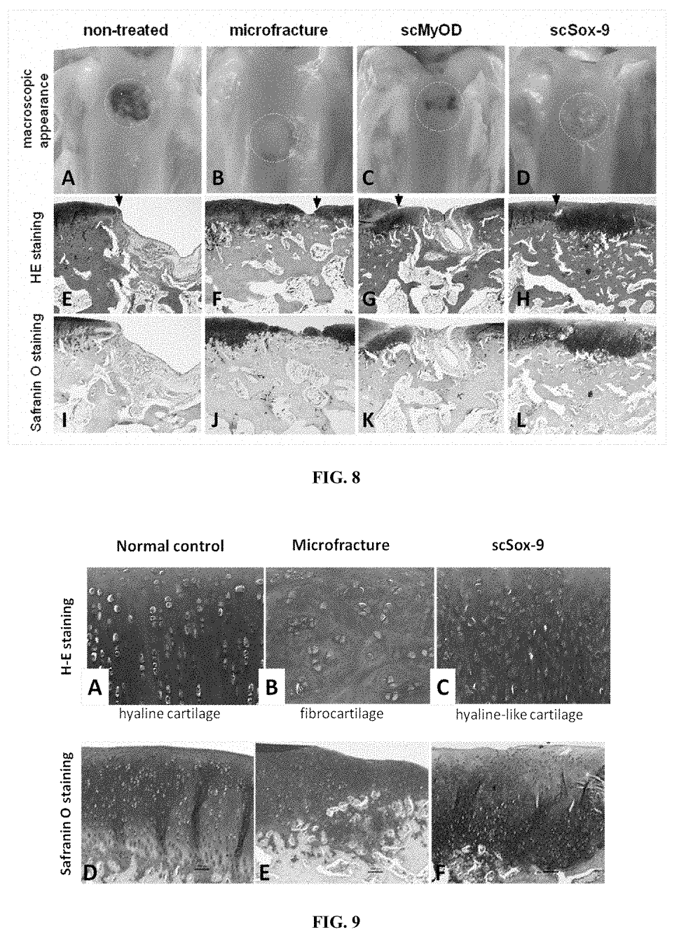

We then tested scSOX9 function in the cartilage defect repair model in New Zealand female rabbits as described. A cartilage defect in full thickness was created in patellar groove of the femur. The defect of cartilage was either left with no treatment, treated with microfracture or microfracture with scSOX9 bound Bio-Gide membrane (FIG. 7). A supercharged recombinant protein, scMyoD bound Bio-Gide membrane was used as control. MyoD is the master transcription factor mediating muscle cell development and skeletal muscle repair. Rabbits were set for free in movement and observed for 8 weeks without restriction of movement. Compared with no treatment or those treated with microfracture only or microfracture with scMyoD, scSOX9 bound Bio-Gide with microfracture treated rabbits showed superior repair of cartilage defect (FIG. 8). Histological analysis revealed that scSOX9 induced regeneration of hyaline cartilage like tissue which is morphologically similar to normal articular cartilage (FIG. 9).

### Example 3

Design, construct and screen cell-penetrating, non-immunogenic SOX9 protein variants for reprogramming MSC to chondrocytes in vitro.

The ability of scGFP fused proteins to efficiently penetrate into cells depends on the strong positive charge of the scGFP moiety, or the fusion proteins' net theoretical charge to molecular weight ratio. Another way to make the SOX9 protein transducible is to add a cell-penetrating peptide (CPP) to SOX9. Upon studying its sequence using the support vector machine (SVM)-based model, an internal putative CPP (177YQPRRRKSVK186) has been identified embedded in SOX9 (Table 1). Therefore, it is possible that the native SOX9 itself is capable of penetrating into cells without the help of the scGFP moiety. Coincidently, this putative CPP also contains the cNLS as shown in FIG. 3.

To further enhance the strength of the putative CPP and improve the ability of SOX9 transduction, a series of SOX9 variants are constructed by initially replacing individual amino acids in the original CPP with positively charged arginine (R) or lysine (K). As shown in Table 1, the confidence levels on the modified CPPs, as represented by the SVM scores calculated using the public online CellPPD tool, increase dramatically compared with the original one (the higher SVM score a peptide has, the more effective it can penetrate into cells). Variants are designed by enhancing CPP and leaving the cNLS intact. Where two or more variants are proved effective in driving SOX9 into cells, more variants are produced to combine the mutations. For example, the internal CPP is changed to 177YQPRRRKRKK186, when Derivative 3 and 4 are both effective.

The best CPP-enhancing SOX9 variant shows weaker transduction capability than scSOX9. To compensate for the potential decrease in protein internalization, a second mutation is introduced to increase SOX9 nuclear retention.

One bottleneck affecting the reprogramming efficiency of transducible transcription factor proteins is that after cell uptake and nuclear translocation, the proteins tend to be pumped back to cytoplasm. A major mechanism of protein nucleocytoplasmic shuttling depends on the Nuclear Export Signal, or NES, a short amino acid sequence within each nuclear protein. SOX9 has an intrinsic NES, with a sequence of 130ELSKYLGKLWRLL142. A mutation disrupting its NES-L138A-abolishes SOX9 export from the nucleus and increases its nuclear retention. Moreover, NES elimination slows down the protein's degradation.

All SOX9 variants are created based on human amino acid sequences. Codons for their genes are optimized for E. coli expression and genes synthesized at GenScript, Inc. An N-terminal cleavable His6 tag is fused to each variant for the purposes of protein purification. The overall strategy for obtaining each refolded protein requires 6 steps: 1) growing E. coli carrying an expression plasmid, 2) inducing the synthesis of the expressed proteins as inclusion bodies, 3) purifying the inclusion bodies with freeze-thaw and detergent washing, 4) solubilizing the inclusion bodies in a 8 M urea buffer, 5) refolding the denatured protein to its native form using our proprietary refolding process, and 6) purifying the refolded protein using sizing column chromatographic procedures to separate correctly refolded protein from its partially or totally unfolded counterparts. The refolding method is tailored for each protein based on a refolding screening using spectrophotometry. Because of the N-terminal poly-histidine tag, Nickel columns are used for both inclusion body and refolded protein purification. The final protein purification is carried out on size exclusion columns and checked on SDA-PAGE gel electrophoresis.

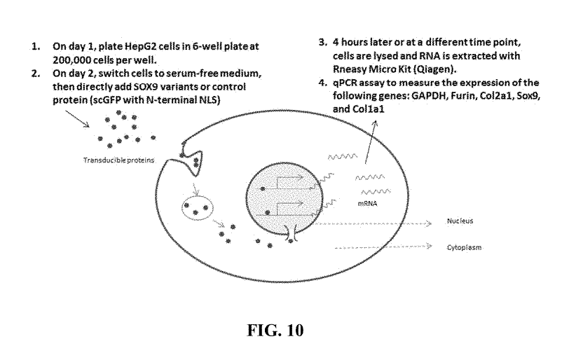

A QC test has been developed for scSOX9 by measuring changes in mRNA expression of several SOX9 target genes (Furin and Col2a1, relative to GAPDH) by qPCR after exposing HepG2 cells to scSOX9 or scGFP in serum-free medium for 4 hours. We use the same assay to screen all the SOX9 variants (FIG. 10). The ability of any SOX9 variant to regulate target genes depends on the protein's strengths in four aspects: 1) its efficiency in penetrating into cells; 2) its efficiency in translocalizing into nuclei; 3) the duration of its nuclear stay; and 4) its transactivating activity. The variants that display the highest activities in activating targeting genes is selected and further optimized in the next round of iteration.

When the best SOX9 format that yields the desired nuclear activities is identified, its capabilities in reprogramming MSCs to chondrocytes are tested. Routine methods such as morphology study, proliferation assay, biomarker staining, and qPCR analysis (FIG. 4-5), are used to evaluate the chondrocytes generated. Differentiation efficiencies is calculated and compared to those produced with the original scSOX9 protein.

TABLE 1
Internal CPP of SOX9 and its enhanced derivatives.
The SVM score indicates how likely each peptide is
a CPP, ranging from 0 to 1, as 1 indicates the
highest probability and strength. (The latter of
amino acid in bold indicates novel mutation to be
created. K = Lysine, R = Arginine.)

| | Sequence | SVM score |
|---|---|---|
| Internal CPP | ₁₇₄YQPRRRKSVK₁₈₃ | 0.62 |
| Derivative 1 | KQPRRRKSVK | 0.87 |
| Derivative 2 | RQPRRRKSVK | 0.88 |
| Derivative 3 | YQPRRRKRVK | 0.91 |
| Derivative 4 | YQPRRRKSKK | 0.90 |

While the invention has been particularly shown and described with reference to specific embodiments (some of which are preferred embodiments), it should be understood by those having skill in the art that various changes in form and detail may be made therein without departing from the spirit and scope of the present invention as disclosed herein.

What is claimed is:

1) A method for repairing cartilage damage comprising:
(a) creating a microfracture or performing similar bone marrow stimulation techniques on a patient inflicted with cartilage damage; and
(b) administering a composition to the site of the microfracture, or to a location accessible to endogenous MSCs, wherein the composition comprises an agent capable of regenerating organized hyaline cartilage.

2) The method of claim 1, wherein the agent is capable of inducing mesenchymal stem cells to differentiate into chondroblasts and/or chondrocytes.

3) The method of claim 2, wherein the agent is a polypeptide.

4) The method of claim 3, wherein the polypeptide comprises an effector domain.

5) The method of claim 4, wherein the effector domain is a transcription factor.

6) The method of claim 5, wherein the transcription factor is SOX9.

7) The method of claim 5, wherein the transcription factor is a variant of SOX9 having an enhanced cell-penetrating peptide.

8) The method of claim 5, wherein the transcription factor is a variant of SOX9 having a disrupted nuclear export sequence.

9) The method of claim 2, wherein the agent is a nucleic acid.

10) The method of claim 9, wherein the nucleic acid encodes a polypeptide comprising a chondrogenic transcription factor.

11) The method of claim 2, wherein the agent is a compound or a small molecule.

12) The method of claim 2, wherein the agent stimulating the expression of SOX9.

13) The method of claim 12, wherein the agent is selected from the group consisting of IGF-1, FGF-2, BMP and TGF-beta.

14) The method of claim 4, wherein the polypeptide further comprises a transduction domain.

15) The method of claim 14, wherein the transduction domain is selected from the group consisting of TAT, poly-arginine, penetratin, VP22, transportan, MAP, MTS, and PEP-1.

16) The method of claim 14, wherein the polypeptide comprises a supercharged peptide.

17) The method of claim 16, wherein the supercharged peptide is supercharged GFP.

18) The method of claim 4, wherein the effector domain is modified to a supercharged form or a transducible format.

19) The method of claim 4, wherein the polypeptide further comprises a ligand of a cell surface receptor.

20) The method of claim 1, further comprising
(c) administering to the patient an immune suppressor.

21) The method of claim 2, wherein the composition further comprises a carrier or a matrix.

22) The method of claim 21, wherein the carrier is a polymer or a PTD peptide.

23) The method of claim 21, wherein the carrier or the matrix is a collagen membrane, or other biocompatible, resorbable membrane, a biocompatible gel, or a fibrin glue.

24) The method of claim 1, wherein the composition is administered by injecting the composition into the synovial cavity where the microfracture is performed.

25) A composition for repairing cartilage damage comprising an agent capable of regenerating organized hyaline cartilage.

26) The composition of claim 25, further comprising a carrier.
